(19) United States
(12) Patent Application Publication
(10) Pub. No.: US 2022/0288308 A1
(43) Pub. Date: Sep. 15, 2022

(54) Blood Infusion System for Controlling Concentration of Calcium in Blood Infusate

(71) Applicant: The Medical College of Wisconsin, Inc., Milwaukee, WI (US)

(72) Inventor: Harvey J. Woehlck, Waukesha, WI (US)

(21) Appl. No.: 17/690,672

(22) Filed: Mar. 9, 2022

Related U.S. Application Data
(60) Provisional application No. 63/159,267, filed on Mar. 10, 2021.

(51) Int. Cl. A61M 5/168; A61M 5/14

(52) U.S. Cl. CPC A61M 5/16827; A61M 5/1408; A61M 5/16831; A61M 2205/18; A61M 2205/502; A61M 2202/0007; A61M 2202/04

(57) ABSTRACT

An infusion pump system is operable to deliver blood to a patient from a first fluid reservoir via a first pump while mixing in a calcium-containing solution from a second fluid reservoir via a second pump. The controller precisely controls the amount of calcium-containing solution that is administered to the patient to be mixed with the blood infusate in the patient's bloodstream according to a preset blood-calcium ratio, thereby preventing citrate intoxication during large blood transfusions.

### BACKGROUND

During massive transfusions, which includes about half of all liver transplants, a few specialty cases like ruptured aortic aneurysm, and some cancer operations as well as many trauma cases, bank blood is given in large quantities to patients. The anesthesiologist or ICU physician, depending on the phase of care, must give calcium chloride or other calcium salts to the patient to prevent fatal citrate intoxication. Most physicians do not know the ratio of calcium to blood which is required, and consequently do not give enough. Many check ionized calcium values during the operation, but without ongoing compensation, this may result in momentary return to normal values, followed by abnormal values that result in hemodynamic instability. Even if physicians know how much calcium to give, it may require dedicated personnel to match the infusion of blood and calcium.

It is more physiologic to give blood as a continuous infusion than as a bolus, and it is difficult to match a bolus of blood to a bolus of calcium because a big bolus of each causes acute electrolyte problems. Giving blood as a continuous infusion requires optimally a continuous infusion of calcium, or intermittent boluses, and therein lies another problem—it takes a person to calculate the amount and to be free to give those, matching the two. When anesthesia personnel are busy, they cannot keep track of the elapsed time, and so the calcium cannot be properly matched.

### SUMMARY OF THE DISCLOSURE

The present disclosure addresses the aforementioned drawbacks by providing a method for controlling an infusion system to infuse blood to a patient. The method includes controlling a first pump of the infusion system via a controller to control a flow rate of blood pumped from a first fluid reservoir by the first pump, and controlling a second pump of the infusion system via the controller to control the flow rate of a calcium-containing solution from a second fluid reservoir by the second pump. The flow rate of the first pump and the flow rate of the second pump are controlled to maintain a preselected ratio of calcium-containing solution to the blood being infused to the patient.

It is another aspect of the present disclosure to provide an infusion pump system that includes a first pump that is configured to pump a first fluid from a first fluid reservoir for infusion into a patient and a second pump that is configured to pump a second fluid from a second fluid reservoir for infusion into the patient. The infusion pump system also includes a controller configured to control a flow rate of the first pump and the second pump, such that the first fluid and the second fluid are provided to a patient in a preselected ratio, where at least one of the first fluid or the second fluid includes blood.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a flowchart setting forth the steps of an example method for controlling an infusion pump system to infuse blood into a patient while controlling the concentration of calcium chloride (or other calcium salt) in the blood infusate.

FIG. 2 is an example blood infusion pump system configured to deliver blood to a patient while precisely controlling the concentration of calcium chloride (or other calcium salt) in the blood infusate.

FIG. 3 is a block diagram of an example controller that is operable to control the blood infusion pump system of FIG. 2.

### DETAILED DESCRIPTION

Described here are infusion pumps and methods for their use. An infusion pump system is provided and operable to deliver blood from a first fluid reservoir of the system to a patient while mixing in a calcium-containing solution from a second fluid reservoir of the system. By precisely controlling the amount of calcium-containing solution that is mixed with the blood infusate according to a preset blood-calcium ratio, the exact amount of calcium (e.g., calcium chloride) in the blood infusate can be controlled in order to prevent citrate intoxication.

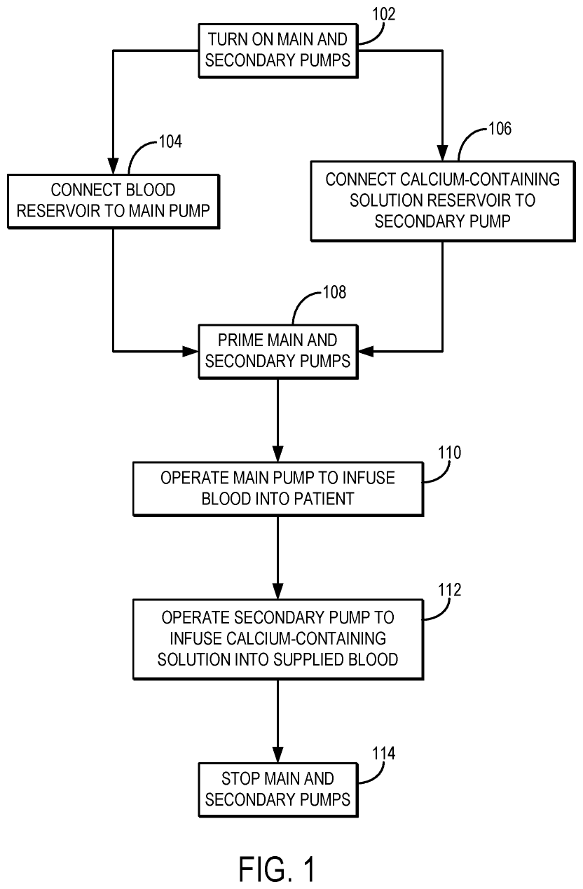

Referring now to FIG. 1, a flowchart is illustrated as setting forth the steps of an example method for controlling an infusion system to deliver blood to a patient while controlling the concentration of calcium in the blood infusate to be at or near a preselected blood-calcium ratio.

The method begins with turning on the main and secondary pumps of the infusion system, as indicated at step 102. For instance, the pumps can be turned on via a control panel or other user interface. After the main pump is turned on, the fluid reservoir containing the blood for infusion is connected or otherwise fluidically coupled to the main pump, as indicated at step 104. Likewise, the fluid reservoir containing the calcium-containing solution is connected or otherwise fluidically coupled to the secondary pump, as indicated at step 106.

Before infusion begins, the main and secondary pumps are preferably primed in order to remove air from the infusion tubing before blood and/or the calcium-containing solution are delivered to the patient, as indicated at step 108. The main and secondary pumps may be manually primed, or alternatively may be automatically primed, as is known in the art.

After the main and secondary pumps have been primed, the main pump is operated in order to deliver blood to the patient, as indicated at step 110. The main pump can be operated in any one of a number of different modes, which may include rapid infusion, bolus infusion, standard infusion, and/or pressure maintenance modes. In general, the main pump is operated according to a preset flow rate that is selected by the user at the control panel or other user interface. The flow rate may be manually or automatically adjusted during operation, as desirable.

While the blood is being delivered to the patient, the secondary pump is also operated to deliver calcium-containing solution to the patient, as indicated at step 112. The secondary pump is operable in response to instructions received from the control panel or other user interface to deliver the calcium-containing solution in proportion to the amount of blood being delivered to the patient. For instance, the secondary pump may be operable to infuse calcium with blood in a ratio specified by the user.

As one non-limiting example, the preset blood-calcium ratio may range from zero up to 1.5 mg calcium chloride (e.g., CaCl₂)) per mL of blood (e.g., per mL of citrated blood product). The ratio may range from 0 mg calcium chloride per mL blood to 1.39 mg calcium chloride per mL blood. In one example, the ratio may be selected from the range of approximately 1.09±0.30 mg calcium chloride per mL blood, or 0.79 mg calcium chloride per mL blood to 1.39 mg calcium chloride per mL blood. In other examples, the ratio may be selected from the range of approximately 0.39 mg calcium chloride per mL blood to 1.39 mg calcium chloride per mL blood. The particular range selected can be determined by the clinical situation and can change with intraoperative events. Preferably, the optimal resulting ionized calcium value will be greater than 0.8 mM and less than 1.4 mM. As still another example, the resulting ionized calcium value can be targeted to be in the range of approximately 1.13 mM to 1.35 mM.

In some implementations, the secondary pump can be manually controlled to adjust the blood-calcium ratio during infusion as desirable. For instance, the user may manually adjust the flow rate of the secondary pump to adjust the volume of calcium-containing solution mixing with the blood being delivered via the main pump. This manual adjustment can be achieved via a control panel or other user interface that forms a part of, or is otherwise in communication with, the infusion system.

Additionally or alternatively, the secondary pump can be automatically or semi-automatically controlled to adjust the blood-calcium ratio during infusion in response to changing clinical conditions. For instance, physiological or other clinical data measured from the patient while the blood is being delivered to the patient can be monitored in order to adjust the amount of calcium-containing solution entering into the patient's bloodstream. As an example, a blood sensor can be used to monitor the concentration of calcium in the blood infusate such that the blood-calcium ratio can be monitored and adjusted in real-time. As another example, after a predetermined amount of blood has been given a visual and/or audible alarm or reminder can be generated and provided to the user to remind the user to draw a blood gas for ionized calcium. This way, the system can provide feedback to the user that labs should be drawn to monitor the blood-calcium ratio.

As indicated at step 114, the main pump and/or secondary pump are then turned off when the desired volume of blood has been infused, under control of the user (e.g., by the user inputting a stop request via a user interface or other input device), or in response to some other preset condition being satisfied.

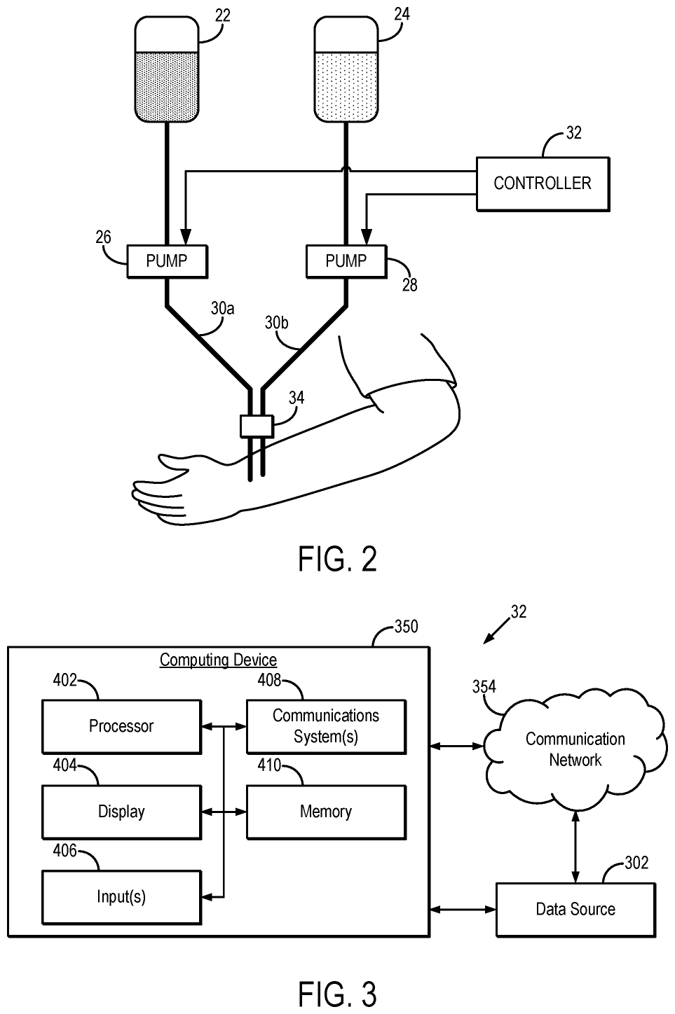

Referring now to FIG. 2, an infusion system 20 that can implement the methods described in the present disclosure is shown. The infusion system 20 generally includes a first reservoir 22 and a second reservoir 24. As an example, the first reservoir 22 may contain blood to be infused to a patient and the second reservoir 24 may contain a calcium-containing solution (e.g., a solution containing CaCl₂) or other suitable calcium salt) to be mixed with the blood as the blood is being infused to the patient. In such examples, the first reservoir 22 may include a blood transfusion bag, or the like, and the second reservoir 24 may include an intravenous (“IV”) fluid bag, a syringe, a tubular reservoir, or the like. Alternatively, the second reservoir 24 may contain a fluid other than a calcium-containing solution. For instance, the second reservoir may contain another solution, compound, agent, or the like that is to be mixed with the patient's blood while the blood is being infused to the patient.

In some embodiments, the infusion system 20 can include a first infusion pump 26 that is fluidically coupled to the first reservoir 22 (e.g., via a first inflow line) and operable to pump fluid from the first reservoir 22 (e.g., via a first outflow line) in order to be infused into the patient. The infusion system 20 can also include a second infusion pump 28 that is fluidically coupled to the second reservoir 24 (e.g., via a second inflow line) and operable to pump fluid from the second reservoir 24 (e.g., via a second outline line) to be infused into the patient. As an example, the first and second infusion pumps 26, 28 can be peristaltic pumps, such as high-speed peristaltic pumps with flow rates that may range from 2.5 mL/min to 1,000 mL/min. In some other embodiments, a single pump may be used, in which case controllable valves may be operable to selectively control the flow of fluid from the first and second reservoirs 22, 24.

The fluid from the second reservoir 24 (e.g., a calcium-containing solution) are preferably mixed with the fluid from the first reservoir 22 (e.g., blood) after being infused into the patient (i.e., the fluids mix in the patient's bloodstream, rather than in the fluid lines 30a, 30b). Advantageously, having the fluid from the first reservoir 22 (e.g., blood) and the fluid from the second reservoir 24 (e.g., a calcium-containing solution) mix only after entering the patient's bloodstream reduces the risk of clotting, which may occur if the fluid from the first reservoir 22 (e.g., blood) and the fluid from the second reservoir 24 (e.g., a calcium-containing solution) mix in the fluid lines 30a, 30b. In some implementations, if the fluid from the first reservoir 22 (e.g., blood) and the fluid from the second reservoir 24 (e.g., a calcium-containing solution) mix are being administered to the patient in a rapid mode, then the fluids may be mixed in the fluid lines 30a, 30b (e.g., by joining or otherwise coupling the fluid lines 30a, 30b into a single fluid line 30 that is provided to the patient) before being infused to the patient, since the rapid mode can reduce the risk of clotting in the fluid lines 30a, 30b.

As described above, fluids from the second reservoir 24 (e.g., calcium-containing solution) are mixed or otherwise combined with fluids from the first reservoir 22 (e.g., blood) in a preselected ratio. Control of the volume of fluid released from the first and second reservoirs 22, 24 is controlled by a controller 32 that is in communication with the first and second pumps 26, 28. In some embodiments, the controller 32 is responsive to measurements of clinical and/or physiological data. For instance, the ratio of fluids from the first and second reservoirs 22, 24 being infused into the patient can be measured and monitored. When the ratio deviates from the preselected ratio, the controller 32 can respond by adjusting the flow rate and/or volume from the first reservoir 22 and/or the second reservoir 24.

In some embodiments, a blood sensor 34 can be in fluid communication with the fluid lines 30a, 30b, such that continuous and/or intermittent monitoring of calcium levels being administered relative to the blood infusate can be performed. A feedback control loop can be entered, in which the level of calcium can be automatically adjusted based on the quantity measured by the blood sensor 34.

The infusion system 20 can be controlled by a user via the controller 32. In general, the controller 32 can include a simple user interface that allows for user control with minimal input selections. As a non-limiting example, the user interface can include a high-contrast display that can be used to provide easy-to-follow on-screen instructions, clear descriptions of alarms and alerts, and quick access to device operations. For instance, the user interface can be operable to allow the user to alter the operating states of the first pump 26 and/or second pump 28 during operation in order to maintain the desired blood-calcium ratio.

The controller 32 can also control other aspects of the infusion system 20 operation. For example, the controller 32 can control a power input to turn on and off the first and second pumps 26, 28. Additionally or alternatively, the controller 32 can control manual and/or automatic priming of the first and second pumps 26, 28.

Responsive to data received by monitoring the pressure of the blood infusate and/or calcium infusate in the fluid lines 30a, 30b, the controller 32 can also control the flow rate of the first pump 26 and/or the second pump 28 in order to adjust the pressure of the blood and/or calcium infusate. For instance, a pressure sensor can be used to monitor the pressure in one or both of the fluid lines 30a, 30b and when the measured pressure exceeds a prescribed threshold the controller 32 can adjust the flow rate in the first and/or second pumps 26, 28, shut off operation of the first and/or second pumps, and/or generate an alarm (e.g., auditory and/or visual alarm) to warn the user of the excessive pressure in the fluid lines 30a, 30b. Similarly, the controller 32 can be operable to maintain the pressure in the fluid lines 30a, 30b at or near a prescribed maintenance pressure.

As described above, the first and/or second pumps 26, 28 can be operable in one or more predefined operation states or modes, such as a rapid infusion mode, a bolus infusion mode, a standard infusion mode, or a pressure maintenance mode. In these instances, the controller 32 can be operable (e.g., by user input via a user interface or other user input) to select the mode of operation for the first and/or second pump 26, 28.

For example, in a standard infusion mode, the first pump 26 may pump blood from the first reservoir 22 at a rate of 120 mL per hour, or in a rapid infusion mode may pump blood from the first reservoir 22 at a rate of 500 mL per minute, or the like. In a bolus infusion mode, the first pump 26 may be operable to deliver blood from the first reservoir 22 in one or more prescribed bolus volumes, such as 100 mL, 250 mL, 500 mL, 750 mL, 1000 mL, and so on. These bolus volumes may be delivered at a predetermined or otherwise selected rate, such as 500 mL per minute. The flow rates and/or bolus volumes in these operation modes can be selected and otherwise adjusted by the user via a user interface or other user input on the controller 32.

Referring now to FIG. 3, an example of a controller 32 for controlling the operation of an infusion system in accordance with some embodiments of the systems and methods described in the present disclosure is shown. As shown in FIG. 3, a computing device 350 can receive one or more types of data (e.g., blood sensor data) from data source 302. In some embodiments, computing device 350 can execute at least a portion of an infusion pump controller system to monitor and control operation of the infusion system from data received from the data source 302, or in response to user input.

In some embodiments, computing device 350 can be any suitable computing device or combination of devices, such as a desktop computer, a laptop computer, a smartphone, a tablet computer, a wearable computer, a server computer, a virtual machine being executed by a physical computing device, and so on.

In some embodiments, data source 302 can be any suitable source of clinical and/or physiological data (e.g., blood sensor measurement data, patient clinical data), such as a blood sensor, another computing device, and so on. In some embodiments, data source 302 can be local to computing device 350. For example, data source 302 can be incorporated with computing device 350. As another example, data source 302 can be connected to computing device 350 by a cable, a direct wireless link, and so on. Additionally or alternatively, in some embodiments, data source 302 can be located locally and/or remotely from computing device 350, and can communicate data to computing device 350 via a communication network (e.g., communication network 354).

In some embodiments, communication network 354 can be any suitable communication network or combination of communication networks. For example, communication network 354 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 354 can be a local area network, a wide area network, a public network (e.g., the Internet), a private or semi-private network (e.g., a corporate or university intranet), any other suitable type of network, or any suitable combination of networks. Communications links shown in FIG. 3 can each be any suitable communications link or combination of communications links, such as wired links, fiber optic links, Wi-Fi links, Bluetooth links, cellular links, and so on.

As shown in FIG. 3, in some embodiments, computing device 350 can include a processor 402, a display 404, one or more inputs 406, one or more communication systems 408, and/or memory 410. In some embodiments, processor 402 can be any suitable hardware processor or combination of processors, such as a central processing unit (“CPU”), a graphics processing unit (“GPU”), and so on. In some embodiments, display 404 can include any suitable display devices, such as a computer monitor, a touchscreen, a television, and so on. As described above, the display 404 can in some configurations generate a display that indicates the amount of calcium-containing solution being administered to the patient, such that the user can monitor this amount. In some other configurations, the display 404 can generate a display that indicates a reminder to the user to perform a blood draw to measure the amount of calcium within the patient's blood. In some embodiments, inputs 406 can include any suitable user interfaces, input devices, and/or sensors that can be used to receive user input, such as a keyboard, a mouse, a touchscreen, a microphone, and so on.

In some embodiments, communications systems 408 can include any suitable hardware, firmware, and/or software for communicating information over communication network 354 and/or any other suitable communication networks. For example, communications systems 408 can include one or more transceivers, one or more communication chips and/or chip sets, and so on. In a more particular example, communications systems 408 can include hardware, firmware and/or software that can be used to establish a Wi-Fi connection, a Bluetooth connection, a cellular connection, an Ethernet connection, and so on.

In some embodiments, memory 410 can include any suitable storage device or devices that can be used to store instructions, values, data, or the like, that can be used, for example, by processor 402 to present content using display 404, to communicate with server 352 via communications system(s) 408, and so on. Memory 410 can include any suitable volatile memory, non-volatile memory, storage, or any suitable combination thereof. For example, memory 410 can include RAM, ROM, EEPROM, one or more flash drives, one or more hard disks, one or more solid state drives, one or more optical drives, and so on. In some embodiments, memory 410 can have encoded thereon, or otherwise stored therein, a computer program for controlling operation of computing device 350. In such embodiments, processor 402 can execute at least a portion of the computer program to present content (e.g., user interfaces, graphics, tables), receive content over the communication network 354, transmit information over the communication network 354, and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A method for controlling an infusion system to infuse blood to a patient, the method comprising:
controlling a first pump of the infusion system via a controller to control a flow rate of blood pumped from a first fluid reservoir by the first pump;
controlling a second pump of the infusion system via the controller to control the flow rate of a calcium-containing solution from a second fluid reservoir by the second pump, wherein the flow rate of the first pump and the flow rate of the second pump are controlled to maintain a preselected ratio of calcium-containing solution to the blood being infused to the patient.

2. The method of claim 1, wherein the ratio of calcium-containing solution in the blood being infused to the patient is such that the blood contains between zero and 1.5 mg of calcium chloride per mL of blood.

3. The method of claim 3, wherein the ratio of calcium-containing solution in the blood being infused to the patient is such that the blood contains between zero and 1.5 mg of calcium chloride per mL of citrated blood product.

4. The method of claim 3, wherein the ratio of calcium-containing solution in the blood being infused to the patient is such that the blood contains between 0.39 mg of calcium chloride per mL of blood and 1.39 mg of calcium chloride per mL of blood.

5. The method of claim 4, wherein the ratio of calcium-containing solution in the blood being infused to the patient is such that the blood contains between 0.79 mg of calcium chloride per mL of blood and 1.39 mg of calcium chloride per mL of blood.

6. The method of claim 1, further comprising measuring a concentration of calcium in the blood being infused to the patient via a blood sensor and adjusting via the controller, the flow rate of the calcium-containing solution from the second fluid reservoir by the second pump in order to maintain the ratio of calcium-containing solution to the blood being infused to the patient.

7. The method of claim 1, further comprising measuring a concentration of calcium in the blood being infused to the patient via a blood sensor and generating an alarm to a user when the concentration of calcium in the blood deviates from the preselected ratio of calcium-containing solution in the blood being infused to the patient.

8. The method of claim 7, wherein the alarm comprises an auditory alarm.

9. The method of claim 7, wherein the alarm comprises a visual alarm.

10. The method of claim 9, wherein the visual alarm comprises a warning generated by the controller and displayed on a user interface of the controller.

11. An infusion pump system, comprising:
a first pump configured to pump a first fluid from a first fluid reservoir for infusion into a patient;
a second pump configured to pump a second fluid from a second fluid reservoir for infusion into the patient;
a controller configured to control a flow rate of the first pump and the second pump, such that the first fluid and the second fluid are provided in a preselected ratio, wherein at least one of the first fluid or the second fluid comprises blood.

12. The infusion pump system of claim 11, further comprising a first inflow line operable to fluidically couple the first pump to the first fluid reservoir, and a second inflow line operable to fluidically couple the second pump to the second fluid reservoir.

13. The infusion pump system of claim 12, further comprising a first fluid line fluidically coupled to the first pump and a second fluid line fluidically coupled to the second pump in order to receive the first fluid and the second fluid when the first pump and the second pump are respectively operating to pump the first fluid from the first fluid reservoir and the second fluid from the second fluid reservoir.

14. The infusion pump system of claim 13, further comprising a sensor coupled to the first fluid line and the second fluid line and being in communication with the controller, wherein the sensor is operable to measure a concentration of the second fluid in the second fluid line and the controller is configured to adjust the flow rate of the second pump responsive to the measured concentration of the second fluid in the fluid line in order to maintain the preselected ratio of the first fluid to the second fluid.

15. The infusion pump system of claim 11, wherein the first pump is a peristaltic pump.

16. The infusion pump system of claim 15, wherein the second pump is a peristaltic pump.

17. The infusion pump system of claim 11, wherein the preselected ratio is between zero and 1.5 mg of calcium chloride per mL of blood.

18. The infusion pump system of claim 17, wherein the preselected ratio is between 0.39 mg of calcium chloride per mL of blood and 1.39 mg of calcium chloride per mL of blood.

19. The infusion pump system of claim 18, wherein the preselected ratio is between 0.79 mg of calcium chloride per mL of blood and 1.39 mg of calcium chloride per mL of blood.
